SEQUENCE LISTING (2 sequences)

SEQ ID NO: 1 - DNA, 3562 bp, Arabidopsis thaliana
aattctgattcaaagcaacgcctctgacgcctgaatcttctggctctctctctttcctcgtgccatcagcgcactatctctagctattgctctctctgcttgtgatcatcattttccccaacggagaacgagagaacgtcgttcgaggaacgaaacggaaagaggttcgtatcgggaggagagagcgaaaatgggggtggaagaaggagctggtgttgataagaagataacgattggagtctgcgtcatggaaaagaaggttttttcagctcccatgggacaaattatggacaggatacatgcgtttggcgaatttgagattatacattttggggacaaggtcatacttgaagacccagtagaaagttggccgatttgtgattgtttgattgctttctattcctctggatatcctcttgagaaagttcaagcatattcttctttaagaaagccctttttagtgaatgaactcgatccccaatatcttcttcatgaccgccggaaagtgtatgagcatcttgagatgtatggtatcccagttcctagatatgcttgtgtcaatagaaaagtaccagacgaagatcttgattatttcgtcgaggaagaagattttgttgaggttaaaggtgaacgattctggaagccatttgtggaaaagcccgtcaatggagatgaccatagtataatgatatactaccctagctcagctggtggaggcatgaaagaattgtttcgtaaggttgggaatcgatcaagtgagtttcatcctgatgtcagaagagtaaggagagaaggttcttatatatatgaggagtttatgcctactgggggaactgatgtcaaggtctacactgtgggtcccgaatatgcacacgctgaagcaagaaaatcacctgttgttgatggtgtagtaatgagaaatccagatgggaaggaagtgagatatccagttttacttacacctgctgagaaacaaatggcgagagaagtttgcattgcatttaggcaagcggtttgcggttttgatctcctacgatctgagggaagttcatatgtttttgacgtaaatggatggagttttgtgaagaactcctataagtattacgacgatgctgcttgtgtgctacggaaaatgtttttggatgcaaaggctcctcatctttcttcaacaattcctcccattctgccatggaaaatcaacgaacctgtccaatctaatgaaggtcttacccgccaaggaagtggcatcatcggaacttttgggcagtcagaggagctacgctgtgtcattgctattgttcggcatggtgatagaacccccaagcagaaagtgaaactaaaagttacagaggagaaactgttaaacctgatgttgaagtacaatggaggaaaaccaagagctgagacaaaacttaaaactgccgtccaattgcaagatctattggatgccacaagaatgttaattcctcgtgcaagatcaggtgaaagtgatagtgatgcagaagaccttgaacacgctgacaagcttcggcaagttaaagccgttcttgaagagggtgggcatttctctggcatatacaggaaggttcaactgaagccgttgaaatgggttaacgtaccaaaaagtgatggtgaaggcgaagaggaacgcccagtggaagctcttatggttctgaaatacgggggtgttctaactcatgctggtagaaagcaggccgaagagcttggtagatactttcgaaacaatatgtatccaggggaaggtaccggtttgctccgtctccatagtacataccgtcacgaccttaaaatttacagctctgacgagggacgtgttcagatgtctgcagctgcttttgccaaaggcctgcttgacctagaaggacagctgaccccaatcctggtttctctggtaagtaaggactcttccatgttggatggccttgataatgccagcagtgaaatggaagcagctaaggctcaactgaatgagatcataaccgccggctcaaagatggtacatgatcacgtctcttctgaattaccttggatgactgatggggctggacttcctcctcacgctgatgagcacctacctgaattggtaaaattagctaaaaaggtgactgaacaagtaaggctacttgcacaagatgaacacgagaatctcgctgagcctagcgcttatgatgtagtccctccatatgatcaagccaaggcccttggcaagtcaaacattgacgttggtcggattgctgccggattaccttgtggtagtgaagggttccttttgatgtttgcccggtggagaaaactcgaaagggatctctacaacgaaagaagagagcggtttgacattacgcagattcctgatgtttacgattcatgcaagtatgacctgttacataattctcatctcgatctaaaaggactagacgaactcttcaaagtagcgcagttgcttgcagatggtgtaatcccaaatgagtatgggatcaatccacagcaaaagctcaaaatcggttcaaagattgcacggcgcctgctgggtaaaatccttattgacttgaggaacactcgagaagaggccatgagtgttgctgaattgaagaacagtcaagatcaagtctcagtgtcattgtattcgtcaagaaaggaggatagatatagtcaaccaaaacttttcgttaaaagcgatgagttgagacggcctagcactggagagaataaagaagaagatgatgataaagaaaccaaatacagactagatccaaagtatgcaaatgtcatgacgcctgaacgtcatgtgaggacacgtctttacttcacatctgaatcacatattcactctctgatgaacgttctacggtattgtaatcttgacgaatctcttcaaggagaagaaagtctggtctgccaaagcgcgttggatcgtctatgtaaaaccaaagaacttgattacatgagctacgttgtcctaaggctgtttgagaacactgagatatctctggacgacccgaaacgatttagaatcgaacttacatttagccgtggcgcagatttgtctcccttagagaagaaggacgaggaagcagaatcattgcttcgagaacacacacttccgataatgggaccagaaaggcttcaagaggttggttcatgtttgaccctcgagacaatggagaagatgatacgtccatttgcaatgccagccgaggatttccctccaccgtgtactccagcaggcttctccggttacttctctaaaagcgccgctgttctcgaacgtcttgttaaactttggcccttccacaagaacacctctaatggcaaaagctaaacgccaacaaagtgggtgactaatgactaatgaatgatgatgatattcctaatccatttattttttgaatcaaaattttgaattgttagtagtttctaattgagtacactaaattgttcattttgttttgtagaatcgtcaaaaaaaattgttaaaaacaattgctaattgatttagcattatggaaaatttccaagcttctacttaaaaaaaaaaaaaaaa

SEQ ID NO: 2 - DNA, 1497 bp, Arabidopsis thaliana
atgtctgcagcggcttttgctaaaggcctgcttgacctagaaggacagctgacgccaatcctggtttctttggttagcaaggactcttccatgttggatggtcttgataatgccagcattgaaatggaagcggccaaggctagattgaatgagattgtaacgtctggcacaaagatgatagacgacgaccaagtctcctctgaagatttcccttggatgactgatggagctggacttcctcccaacgctcatgaactcctacgtgaactggtgaaattaactaagaatgtgactgaacaagtaagactacttgcaatggatgaagacgagaacctcactgagccatacgatataattcctccatatgatcaagcaaaagcccttggcaagacaaacattgacagtgatcggattgcttctggattaccatgtggtagtgaaggattccttctgatgtttgctcggtggataaaacttgctagggatctctacaatgaaagaaaagaccgatttgacatcacacagattccggatgtttacgattcatgcaagtacgacctgttacataattcccatctcgatctaaaaggattagatgaactcttcaaagtagcacagttacttgcagatggtgtaatcccaaatgagtatggcatcaatccgcaacaaaagcttaaaatcggttcaaagattgctcggcgcttaatggggaaaatcttgatagacttgaggaatactcgagaagaagcactgagcgttgccgagttgaaagaaagccaagaacaagtcttgtcattatccgcctcgcaaagggaagatagaaatagtcaaccgaagctatttatcaatagcgatgaattgagacgacctggcacaggtgataaagatgaagatgatgataaagaaaccaaataccgattagatccaaagtatgcaaatgtgaagacacctgaacgtcatgtgaggactcgactttacttcacatctgaatcgcatattcattcgctcatgaacgtccttcgatactgtaaccttgatgaatctctcctaggagaagaaagcctcatttgccaaaacgccttagaacgtctctgtaaaaccaaggaactcgattacatgagttacattgtcctaaggctctttgagaacaccgaggtatcacttgaagacccgaaaagattccgcattgaacttacatttagccgtggagctgatttgtctcccttaaggaataatgacgacgaggcagagacattgctaagggaacacacacttccgataatgggaccagagaggcttcaagaagtaggttcttgtttaagcttggagacaatggagaaaatggttcgtccatttgctatgccggcggaagatttccctccggcgtccactccggttggtttctcgggttacttctcaaaaagtgctgcggtgctggagcgactcgtaaacctcttccacaactataagaactcttcttctaatggaaggagctaa

(19) United States
(12) Patent Application Publication
(10) Pub. No.: US 2017/0306350 A1
(43) Pub. Date: Oct. 26, 2017

(54) PLANTS HAVING INCREASED RESISTANCE TO PLANT PATHOGENS, AND METHOD FOR CREATING INCREASED PATHOGEN RESISTANCE IN PLANTS

(71) Applicant: Eberhard Karls Universität Tübingen, Tübingen (DE)

(72) Inventors: GABRIEL SCHAAF, Tübingen (DE); DEBABRATA LAHA, Tübingen (DE); MARC FREYER, Dettenhausen (DE); MARÍLIA K.F. DE CAMPOS, Tübingen (DE); PHILIPP JOHNEN, Tübingen (DE); SASKIA C.M. VAN WEES, MAARSSEN (NL)

(73) Assignee: Eberhard Karls Universität Tübingen, Tübingen (DE)

(21) Appl. No.: 15/523,637

(22) PCT Filed: Nov. 5, 2015
(86) PCT No.: PCT/DE2015/000534

§ 371 Date: May 1, 2017

(30) Foreign Application Priority Data
Nov. 12, 2014 (DE) 10 2014 016 774.7

(51) Int. Cl. C12N 15/82; C07K 14/415; A01N 57/12

(52) U.S. Cl. CPC C12N 15/8279; C12N 15/8286; C12N 15/8282; C12N 15/8245; A01N 57/12; C12N 15/8234; C12N 15/8225; C07K 14/415; C12N 15/8235

(57) ABSTRACT

The invention relates to plants with increased resistance to plant pathogens, wherein the intracellular concentration of inositol pyrophosphate InsP₇ and/or InsP₈ in said plants is increased in comparison to the wild-type plant. In particular, the invention involves plants with increased expression of at least one protein involved in the synthesis of inositol pyrophosphates InsP₇ and/or InsP₈, such as, for example, proteins VIH2 and VIH1. The plants according to the invention are particularly resistant to the following plant pathogens: herbivore insects, for example larvae of agriculturally relevant pests, pathogenic fungi, such as necrotrophic fungi, or other plant pests, such as biotrophic pathogens. The invention further relates to the method for increasing plant resistance to plant pathogens, wherein the intracellular concentration of inositol pyrophosphates InsP₇ and/or InsP₈ is increased in comparison to the wild-type plant.

### CROSS-REFERENCE TO RELATED APPLICATIONS

This U.S. non-provisional patent application is a U.S. national stage application, which was filed on May 1, 2017 under 35 U.S.C. §371 and claims priority to PCT Patent Application No. PCT/DE2015/000534, which was filed on Nov. 5, 2015, and to German Patent Application No. DE 10 2014 016 774.7, which was filed on Nov. 12, 2014. The contents of PCT Patent Application No. PCT/DE2015/000534 and German Patent Application No. DE 10 2014 016 774.7 are incorporated herein by reference in their entirety.

### SEQUENCE LISTING

This application includes a Sequence Listing in electronic format as a txt file entitled “EKUT-01-0103USWO_2017-05-01_U3077WOUS_US15523637_SEQLIST_ST25,” which was created on May 1, 2017 and which has a size of 7,230 bytes. The contents of txt file “EKUT-01-0103USWO_2017-05-01_U3077WOUS_US15523637_SEQLIST_ST25” are incorporated by reference herein.

### DESCRIPTION

The present invention relates to plants having increased resistance to plant pathogens as well as the method for producing increased pathogen resistance in plants by influencing the intracellular concentration of inositol pyrophosphates, in particular of InsP₈.

Plants are constantly exposed to plant pathogens. In particular, herbivorous insects and their larvae, as well as fungi and fungi-like pathogens (e.g. oomycetes). Conservative estimates propose that the agronomic damage caused by Botrytis species alone is estimated to be around 1 Billion/year¹. The resulting damages are caused not only by immediate harvest loss, but also by the loss of quality of products (for example by enrichment of mycotoxins).

A conventional method for fighting off insects and fungal pathogens is the use of chemical plant protection products. However, these measures often result in a loss of a part of the yields. The use of chemical plant protection products in crops can have a negative impact on people and the environment. Furthermore, these measures are very cost- and labor-intensive. Moreover, the pathogens to be combated with chemical plant protection products frequently develop adaptation mechanisms so that such measures often do not achieve the desired protective effect.

An alternative to the use of chemical agents is the use of insect and fungus resistant varieties. A breeding of new varieties using conventional plant breeding is very tedious and difficult because of the complexity of plant genomes; conventional plant cultivation usually uses spontaneous or induced mutations, the manifestations of which are not influenced by external factors (eg. cold-shocks or radioactive irradiation).

An alternative to conventional breeding is the production of genetically modified plants. Genes with desired properties are specifically introduced into the genome of the plants. Currently, in particular herbicide- and insect-resistant plants are marketed as genetically modified plants. The goal of the new generation of varieties is the increase in yield under adverse conditions, such as dry stress or insect infestation.

Mostly genetically modified maize and cotton plants with insect resistance are currently being cultivated. In most cases this property is derived from the Bt toxin, which is encoded by a gene introduced into the plants. The gene originates from the soil bacterium Bacillus thuringiensis, which naturally produces this active ingredient. One of the problems of this strategy is, however, that the first resistances have already developed in plant pathogens.

Furthermore, many scientific papers have shown that the Bt maize harms butterflies and endangers numerous other non-target organisms. Bt toxins are only slowly degraded, accumulate in the soil and are passed on in the food chain. Similar to the use of Bt toxins in biologic plant protection, as well as the control of mosquitoes, the cultivation of Bt maize therefore entails a wide range of risks to biodiversity.

The object of the present invention is, therefore, to provide plants with elevated resistance to plant pathogens, as well as processes for the preparation thereof, which do not harbor the disadvantages of the long-term toxicity of active substances known from the prior art, for example, by accumulation in the soil. Moreover, by providing such plants, and methods respectively, the spectrum of possible measures against pathogens shall be expanded and thereby the probability of resistance development be minimized.

This object is achieved by providing plants in which the intracellular concentration of inositol pyrophosphates InsP₇ and/or InsPe₈ is increased in comparison to a wildtype plant.

In animal systems, inositol pyrophosphates have been described as important intracellular signaling molecules. In the present invention, it was shown for the first time that the proteins VIH2 and VIH1 inhibit the phosphorylation of inositol pyrophosphates InsP₆ and InsP₇, and that VIH2 in Arabidopsis seedlings is mainly responsible for the synthesis of the inositol pyrophosphate InsP₈. The VIH2 transcript is primarily expressed in different vegetative tissues and induced by mechanical injury as well as by infestation by caterpillars, while the VIH1 transcript accumulates mainly in pollen.

In the present invention, it was shown that VIH2 (GenBank Accession: At3g0130) is involved in pathogen defense in plants (see examples).

The object of the present invention is achieved, in particular, by the provision of a plant with inducible or elevated expression of at least one protein which is involved in the synthesis of inositol pyrophosphates, in particular of InsP₈.

One embodiment of the invention is a plant in which the expression and/or the activity of the protein VIH2 encoded by the nucleotide sequence 2 (GenBank Accession: At3g01310), or of a homologous protein which is capable of synthesizing inositol pyrophosphates, in particular InsP₈, is inducible or is increased in the whole plant or in specific tissues in comparison with the wildtype plant.

In another embodiment of the invention, the plant is a plant in which the expression and/or the activity of the protein VIH1 encoded by the nucleotide sequence 1 (GenBank Accession: At5g 15070), or a homologous protein which is capable of synthesizing inositol pyrophosphates, in particular InsP₈, is inducible or is increased in the whole plant or in specific tissues in comparison with the wildtype plant.

The nucleotide sequence 1 or the nucleotide sequence 2 can originate from one plant species, i.e. are expressed homologous, or are derived from another organism, i.e. are expressed heterologously. The heterologous expression can be of advantage, since post-transcriptional or post-translational regulatory mechanisms in the host organism (for example deactivation of the enzyme due to overproduction) can be circumvented frequently.

The inducibility of VIH2, VIH1 or its homologous proteins can be achieved by methods known to those skilled in the art. For example, the expression of corresponding nucleotide sequences can be achieved under the control of an inducible promoter in the target plant. Known inducible expression systems that have already been successfully used in Arabidopsis, tobacco, rice, or maize normally consist of two components: a (often chimeric) transcription factor that is constitutively or tissue-specific expressed and the actual promoter that controls the expression of the desired nucleotide sequence. This promoter can be activated by the chimeric transcription factor by an external stimulus. Known examples are ethanol-inducible (“AlcR/AlcA”-system), dexamethasone-inducible (GR-fusions, GVG- and pOp/LhGR-systems), β-estradiol-inducible (XVE/OlexA system) and heat shock-inducible expression systems.

For the induced expression, promoters naturally occurring in the target plant, which are, for example, induced by pathogens, could also be used. Known examples are, for example, promotors which regulate the expression of the transcripts of the JAZ (“jasmonate ZIM-domain”) proteins which are important in the jasmonate metabolism and occur in all higher plants.

The increased expression (over-expression) of VIH2, VIH1 or its homologous proteins can be achieved by methods known to those skilled in the art. Thus, the expression of corresponding nucleotide sequences can be carried out under the transcriptional control of a constitutive promoter, for example the cauliflower mosaic virus promoter CaMV 35S or an ubiquitin (UBQ) promoter. It is also possible to use tissue-specific promoters which are, for example, expressed only in the tissues potentially infected by pathogens, such as, leaf, fruit or seed specific promoters.

The invention also relates to a method for increasing plant resistance against plant pathogens, wherein the intracellular concentration of inositol pyrophosphates InsP₇ and/or InsP₈, is modulated or is increased in comparison with the wildtype plant.

The intracellular concentration of inositol pyrophosphates InsP₇ and/or InsP₈ can be achieved, in particular, by inducible or constitutive expression and/or activity of VIH2, VIH1 or their homologous proteins.

In an alternative embodiment, the intracellular concentration of inositol pyrophosphates InsP₇ and/or InsP₈, is increased by treating the plants with the substrate for InsP₇ (the precursor of InsP₈), with InsP₈ and/or with InsP₇ or InsP₈ derivatives, for example in the form of spraying, sprinkling or the like. In this case, membrane permeable esters are of particular interest, for example, those that have been developed for the exogenous application of the messenger InsP₃_{²}. In this case, the activity of esterases occurring naturally in the cell is utilized, which after uptake of the derivative leads to a release of the active inositol phosphate or of the inositol pyrophosphate.

The plant pathogens against which the plant according to the invention is resistant are, in particular, herbivore insects, for example larvae of agricultural relevant pests, such as the small cabbage white or the owlet moth, as well as pathogenic fungi, such as necrotrophic fungi, for example representatives of the genera Alternaria or Botrytis.

The advantage of the present invention in contrast to the methods known from the prior art, for example, the expression of the Bt toxin from the bacterium Bacillus thuringiensis in plants, is that in the plant according to the invention endogenous mechanisms can be advantageously utilized for increasing resistance to plant pathogens and environmental stress. Since this entails the use of original plant genes and not of foreign species genes and substances, it can be assumed that the acceptance of such plants in the public is better than it is the case with conventional genetically modified plants. Moreover, it is not expected that the agricultural use of plants according to the invention will have a serious impact on non-target organisms and on the environment, such as the use of conventional insecticides or the cultivation of Bt plants. It can also be assumed that the resistance formation of pests, as opposed to conventional methods for the production of resistant plants, occurs more slowly, since the whole plant defense machinery against pathogens is induced by inositol pyrophosphates and not just individual toxins.

Further advantages, features and possible applications of the invention are described in the following with reference to the below described exemplary embodiment referring to the figures.

FIG. 1: Arabidopsis VIH2 loss of function mutants show a reduced resistance to larvae of the small cabbage white (Pieris rapae). The larvae development was investigated in a so-called ‘no choice’ assay. In this assay, one single caterpillar in larvae stage L1 is placed on a single 5-week-old plant of the indicated genotype, and prevented from escaping using gauze. Col-0 denotes the used wildtype Arabidopsis thaliana (thale cress) ecotype, vih2-3 and vih2-4 are VIH2 loss of function mutants in Col-0 background. The fresh weight of the caterpillars was determined after 7 days. The values indicate the mean value (±standard error) (n=20). Statistically significant differences are indicated by an asterisk (t-test; *p<0.02).

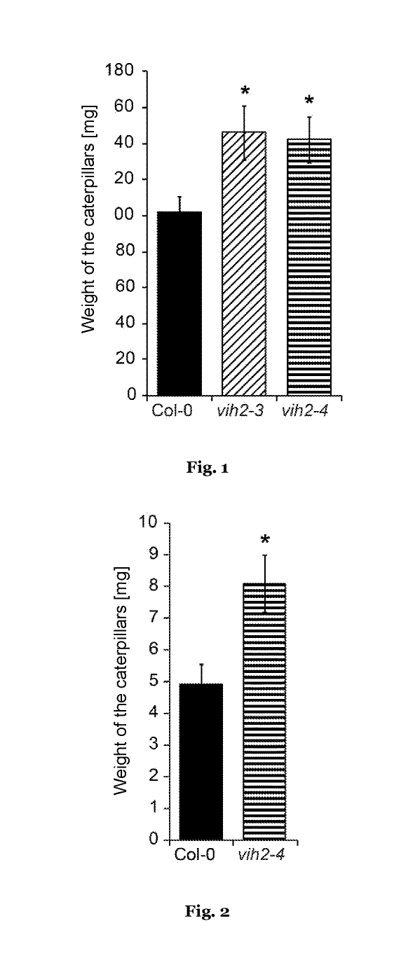

FIG. 2: Arabidopsis VIH2 loss of function mutant shows a reduced resistance to larvae of the owlet moth Mamestra brassicae (cabbage moth). The Larvae development was investigated in a so-called ‘no choice’ assay. In this assay, one single caterpillar in larvae stage L1 is placed on a single 5-week-old plant of the indicated genotype, and prevented from escaping using gauze. Col-0 denotes the used wildtype Arabidopsis thaliana (thale cress) ecotype, vih2-4 is a VIH2 loss of function mutant in Col-0 background. The fresh weight of the caterpillars was determined after 8 days. The values indicate the mean value (±standard error) (n=20). Statistically significant differences are indicated by an asterisk (t-test; *p<0.02).

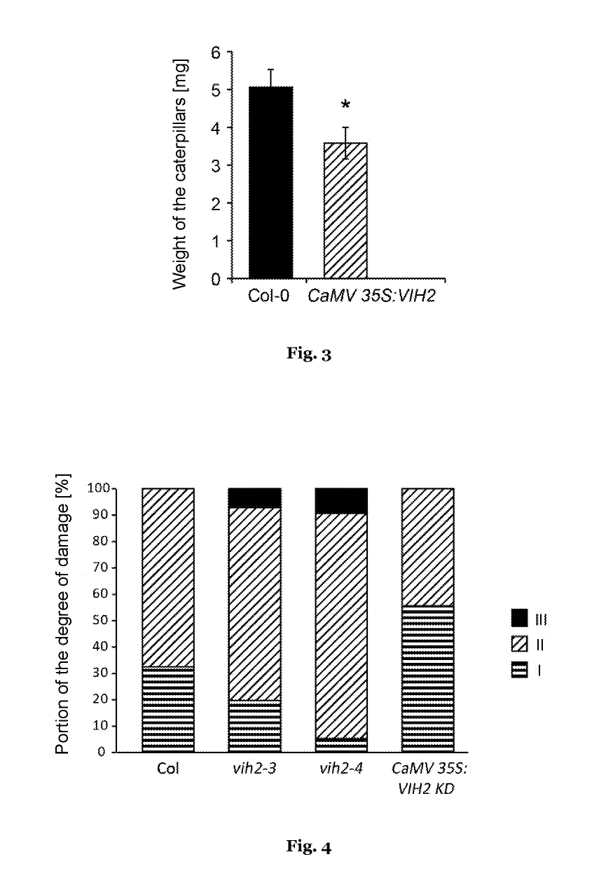

FIG. 3: Arabidopsis VIH2 overexpressing lines show increased resistance to larvae of the owlet moth Mamestra brassicae (cabbage moth). The Larvae development was investigated in a so-called ‘no choice’ assay. In this assay, one single caterpillar in larvae stage L1 is placed on a single 5-week-old plant of the indicated genotype, and prevented from escaping using gauze. Col-0 denotes the used wildtype Arabidopsis thaliana (thale cress) ecotype, “CaMV 35S: VIH2” are transgenic plants in which the kinase domain of the wild-type VIH2 gene is overexpressed under the control of the strong viral CaMV 35S promoter. The fresh weight of the caterpillars was determined after 8 days. The values indicate the mean value (±standard error) (n=20). Statistically significant differences are indicated by an asterisk (t-test; *p<0.05). These experiments show that an increase in the expression of VIH2 (which is linked to an increase in the inositol pyrophosphate InsP₈) leads to an increased resistance of the plants to herbivorous insect pests. The growth of pests on the transgenic plants (and in approximation of pest-induced damage) is reduced by approx. 30%. There are no “undesirable” side effects of the inositol pyrophosphate increase: the plants are healthy and develop and reproduce normally.

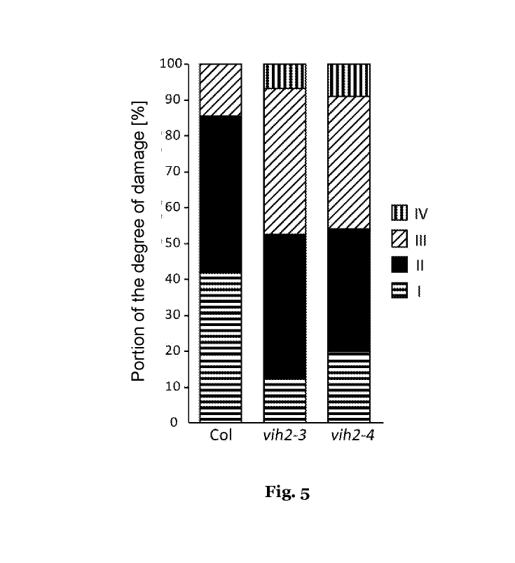

FIG. 4: In Arabidopsis, over-expression of VIH2 leads to increased resistance to the necrotrophic ascomycete Alternaria brassicicola, whereas VIH2 loss of function mutants show reduced resistance. Infection experiments with Alternaria brassicicola (isolate MUCL 20297) were carried out as described previously³. For this purpose, spores were adjusted to a density of 5×10⁵ spores/ml, four to six 5 μl drops of the spore suspension were applied to the leaf surface, and the plants were incubated at 100% humidity, at 22° C. and an 8-hour/16 hours light/darkness-rhythm for 7-10 days (number of plants≧15). Subsequently the disease symptoms were documented and divided into different classes for each phenotype of leaf damage. The classes were as follows. Class I (cross-striped): light brown spots on the infection site; class II (oblique striped): dark brown spots on the infection site and first signs of necrosis; class III (consistently black): progressive necrosis and leaf maceration. The distributions of the data were evaluated with a Chi-square test and showed that the differences between Col-0 and vih2-3, between Col-0 and vih2-4, as well as between Col-0 and CaMV 35S: VIH2 are significant (p<0.05). Vih2-3 and vih2-4 are VIH2 loss of function mutants, CaMV 35S: VIH2 are transgenic plants in which the kinase domain of the wild-type VIH2 gene is overexpressed under the control of the strong, viral CaMV 35S promoter. These experiments show that an increase in the expression of VIH2 (which is associated with an increase in the inositol pyrophosphate InsP₈) leads to an increased resistance of the plants against nectrotrophic fungi, since the fungus-induced damage on such plants is significantly reduced.

FIG. 5: Arabidopsis VIH2 loss of function mutants show a reduced resistance to the necrotrophic fungus and causative agent of the grey mould Botrytis cinerea. A 5 μL large drop of a conidia (spore) suspension of Botrytis cinerea was pipetted onto the leaf surface of a 5-week-old plant. This was done for 5 grown leaves per plant and a total of 20 plants of the indicated genotype. Only leaves younger than the 4^{th} leaf of the respective plant were used. Thereafter, the inoculated plants were incubated at 100% humidity for 3 days at 21° C. in a climatic chamber under a 10-hour/14-hour light/dark-rhythm. Subsequently, the disease symptoms were documented and divided into different classes according to the size and the time of occurrence of lesions. The classes were as follows: class I (cross-striped): lesions of 2 mm diameter; class II (continuous black): lesions of 2 mm diameter with chlorosis; class III (oblique striped): lesions of 2-4 mm diameter with chlorosis; class IV (longitudinally striped): lesions with a diameter >4 mm and chlorosis. The distributions of the data was done with a chi-square test and show that the differences between Col-0 and vih2-3 as well as between Col-0 and vih2-4 are significant (p<0.001). The conidial suspensions for these experiments were prepared as described previously⁴. For this purpose, conidia from Botrytis cinerea were inoculated from a glycerol stock to semi-concentrated potato dextrose broth solid medium (PDB, Difco™) and incubated for 2 weeks at 22° C. under a 10-hour/14-hour light/dark-rhythm. Subsequently, conidia were washed from the surface of the solid medium with semi-concentrated PDB liquid medium, filtered through glass wool, and the conidia-density was determined with a counting chamber. The suspension was adjusted in semi-concentrated PDB-liquid medium to a density of 5×10⁵ conidia/ml, incubated at room temperature for 2 hours and used for leaf inoculation. The experiments were repeated 3 times with similar results.

### EXEMPLARY EMBODIMENTS

The experiments were performed with isogenic lines of the same ecotype (Arabiopsis thaliana, Col-0), which are characterized by presence (Col-0) and absence (vih2-3 and vih2-4) of an intact VIH2 gene (and thus VIH2 protein), or by increased expression of the VIH2 kinase domain (CaMV 35S: VIH2). In the latter plants (CaMV 35S: VIH2), the kinase domain of the wild-type VIH2 gene was under the control of the strong viral CaMV 35S promoter. For this purpose, the VIH2 kinase sequence was amplified by an Arabidopsis cDNA and inserted into the vector pENTR™/D-TOPO® (Invitrogen Life Technologies). From there the VIH2 kinase domain sequence was transferred by Gateway® LR Clonase™ II (Invitrogen Life Technologies) into the binary plant transformation vector pGWB441 (Nakagawa et al., 2007, Biosci. Biotechnol. Biochem, 71, 2095-2100). The vector produced thereby “pGWB441-VIH2 KD” was used for the transformation of Arabidopsis plants. Several independent transformants were selected on kanamycin, no longer segregating CaMV 35S: VIH2 T3 plants were established and an increased InsP₈ biosynthesis was confirmed in these plants. FIGS. 3 and 4 show examples of experiments with one of these lines demonstrating that the increased expression of the VIH2 kinase domain (and accompanying increased production of InsP₈) leads to increased resistance to both larvae of the herbivorous insect Mamestra brassicae (cabbage moth, FIG. 3) as well as against the necrotrophic fungus Alternaria brassicicola (FIG. 4). The used VIH2 loss of function plants (vih2-3 and vih2-4) originate from publically accessible seed repositories as follows: Arabidopsis thaliana Col-0 (Columbia-0, CS60000 whose genome is completely sequenced) from the ‘Salk Institute Genomic Analysis Laboratory’ (USA); Vih2-3 (SAIL_165_F12) from the Syngenta Arabidopsis Insertion Library (SAIL) collection. This line was made available to us through the ‘Arabidopsis Biological Research Center’ (ABRC) of Ohio State University’ (USA); Vih2-4 (GK-080A07) originates from the collection “Genomanalyse im biologischen System Pflanze (GABI-KAT)” of the university Bielefeld. In both cases (SAIL and GABI-KAT), Arabidopsis thaliana Col-0 plants were transformed by transformation with specific T-DNA-containing vectors with the aid of agrobacteria. The T-DNAs integrate largely un-directed into the genome and, depending on the genomic locus, can lead to the destruction of the affected gene (and thus the loss of the protein encoded by this gene). A plurality of such plants are sequenced in the corresponding repositories with the aid of T-DNA-specific oligonucleotides in order to determine the insertion site and thus the identity of the affected gene in a specific plant. Corresponding data are made available online in order to enable the identification of a potential loss of function mutant of the desired protein. This detour (non-directed insertion and subsequent genotyping) is necessary, since in a higher plant a targeted generation of ‘knockout’ plants by the principle of homologous recombination (in contrast to, for example, baker's yeast or mice) is very ineffective.

The exemplary embodiments indicate that VIH2 loss of function mutants have reduced resistance to herbivorous insects and nectrotrophic fungi (FIGS. 1, 2, 4 and 5), whereas the increased expression of VIH2 kinase domain (and thus the increased production of InsP₈) leads to an increased resistance to herbivorous insects and nectrotrophic fungi (FIGS. 3 and 4).

### REFERENCES

1. Dean R, Van Kan J A, Pretorius Z A, Hammond-Kosack K E, Di Pietro A, Spanu P D, et al. The top 10 fungal pathogens in molecular plant pathology. Mol Plant Pathol 2012, 13 (4): 414-430.
2. Dakin K, Li W H. Cell membrane permeable esters of D-myo-inositol 1, 4,5-trisphosphates. Cell calcium 2007, 42 (3): 291-301.
3. Kemmerling B, Schwedt A, Rodriguez P, Mazzotta S, Frank M, Abu Qamar S, et al. The BRIT-associated kinase 1, BAK1, has a Brassinoli-independent role in plant cell death control. Current Biology, 2007, 17 (13): 1116-1122.
4. Submit Corrections Close Van Wees S C, Van Pelt J A, Bakker P A, Pieterse C M. Bioassays for assessing jasmonate-dependent defenses triggered by pathogens, herbivorous insects, or beneficial rhizobacteria. Methods Mol Biol 2013, 1011: 35-49.

What is claimed is:

1. A plant with increased resistance to plant pathogens, in which the intracellular concentration of inositol pyrophosphates InsP₇ and/or InsP₈ is increased in comparison with the wildtype plant.

2. The plant as claimed in claim 1, having inducible or increased expression of at least one protein involved in the synthesis of inositol pyrophosphates InsP₇ and/or InsP₈.

3. The plant as claimed in claim 1, wherein the expression and/or the activity of a protein selected from the group consisting of VIH2 encoded by a nucleotide sequence 2 (GenBank Accession: At3g01310), VIH1 encoded by a nucleotide sequence 1 (GenBank Accession: At5g15070), and a homologous protein capable of synthesizing inositol pyrophosphates InsP₇ and/or InsP₈, is in the whole plant or in specific tissues inducible, or is increased in comparison with the wildtypes.

4. (canceled)

5. The plant according to claim 3, in which the nucleotide sequence 1 or the nucleotide sequence 2 originates from the same plant species or from a different organism.

6. The plant according to claim 3, wherein the nucleotide sequence 1 or the nucleotide sequence 2 is under the control of a promoter that is selected from the group consisting of an inducible promoter and a constitutive promoter.

7. (canceled)

8. The plant according to claim 6, wherein the promoter is tissue-specific, for example leaf-, fruit- or seed-specific.

9. The plant according to claim 1, wherein the plant pathogens are herbivorous insects, for example larvae of agriculturally relevant pests, such as the small cabbage white or the owlet moth, or pathogenic fungi, such as necrotrophic fungi, for example, representatives of the genera Alternaria or Botrytis, or other plant pests, including biotrophic pathogens.

10. A method for increasing plant resistance against plant pathogens, wherein the intracellular concentration of inositol pyrophosphates InsP₇ and/or InsP₈ is increased in comparison to the wildtype plant.

11. The method according to claim 10, wherein the plants are treated with InsP₇, with InsP₈ and/or with InsP₇ or InsP₈ derivatives, for example in form of sprinkling, spraying or the like.

12. The method according to claim 11, wherein the derivatives are membrane permeable esters.

13. The plant as claimed in claim 2, wherein the expression and/or the activity of a protein selected from the group consisting of VIH2 encoded by a nucleotide sequence 2 (GenBank Accession: At3g01310), VIH1 encoded by a nucleotide sequence 1 (GenBank Accession: At5g15070), and a homologous protein capable of synthesizing inositol pyrophosphates InsP₇ and/or InsP₈, is in the whole plant or in specific tissues inducible, or is increased in comparison with the wildtypes.

14. The plant according to claim 13, in which the nucleotide sequence 1 or the nucleotide sequence 2 originates from the same plant species or from a different organism.

15. The plant according to claim 13, wherein the nucleotide sequence 1 or the nucleotide sequence 2 is under the control of a promoter that is selected from the group consisting of an inducible promoter and a constitutive promoter.

16. The plant according to claim 14, wherein the nucleotide sequence 1 or the nucleotide sequence 2 is under the control of a promoter that is selected from the group consisting of an inducible promoter and a constitutive promoter.

17. The plant according to claim 15, wherein the promoter is tissue-specific, for example leaf-, fruit- or seed-specific.

18. The plant according to claim 16, wherein the promoter is tissue-specific, for example leaf-, fruit- or seed-specific.

19. The plant according to claim 2, wherein the plant pathogens are herbivorous insects, for example larvae of agriculturally relevant pests, such as the small cabbage white or the owlet moth, or pathogenic fungi, such as necrotrophic fungi, for example, representatives of the genera Alternaria or Botrytis, or other plant pests, including biotrophic pathogens.

20. The plant according to claim 3, wherein the plant pathogens are herbivorous insects, for example larvae of agriculturally relevant pests, such as the small cabbage white or the owlet moth, or pathogenic fungi, such as necrotrophic fungi, for example, representatives of the genera Alternaria or Botrytis, or other plant pests, including biotrophic pathogens.
